SEQUENCE LISTING (16 sequences)

SEQ ID NO: 1 - DNA, 738 bp, Streptococcus suis
  source 1..738: mol_type = genomic DNA
atgacaacagtaaatgaagcattaaataatgtaagagctcaggttgggtccggtgtgtctgttggcaacggcgaatgctacgctttggctagttggtacgagcgcatgattagtccggatgcaactgtcggacttggcgctggtgtgggctgggtcagcggtgcaatcggcgatacaatctctgccaaaaacatcggctcatcatacaactggcaagctaacggctggacagtttccacatctggtccatttaaagcaggtcagattgtgacgcttggggcaacaccaggaaacccttacggacatgtggtaatcgtcgaagcagtggacggcgatagattgactattttggagcaaaactacggcgggaaacgttatcccgtccgtaattattacagcgctgcaagctatcgtcaacaggtcgtgcattacatcacaccgcctggcacggtcgcacagtcagcacccaaccttgcaggctctcgttcctatcgcgagacgggcactatgactgtcacggtcgatgctctcaatgttcgcagggcgccaaatacttcaggcgagattgtagcagtatacaagcgtggtgaatcatttgactatgatactgtcatcatcgatgtcaatggctatgtctgggtgtcttacataggcggcagcggcaaacgtaactacgttgcgacgggcgctaccaaagacggtaagcgtttcggcaatgcttggggtacatttaaataa

SEQ ID NO: 2 - protein, 280 aa, unidentified
  REGION 1..280: note = source = /note="Description of Unknown:ClyS lysin sequence"
METLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNMVLQGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 3 - protein, 288 aa, unidentified
  REGION 1..288: note = source = /note="Description of Unknown:PlyV12 lysin sequence"
MTRRYTKMNVPQSLVNWFVNHRNLLTYSMYGSRNGSDGTADCSGSMSQALKEAGIPIQGLPSTVTLGQQLAKNGFYRISRNEDWNAETGDIVLMSWGADMASSGGAGGHVGVMMDSVNFISCDYSTQGAAGQAINTYPWNDYYEANKPAYIEVWRYSESAPQTKNQANTAVTPQQKAYYEANEVKYVNGIWQIKCDYLSPIGFDYLENGIPVTMVNWVDKDGNDLPDGADQDLKAGMYFSFSSDETNIVDTGNGGYYGGYYWRLFEFGQFGPVWLSCWNKDDLVNYFQ

SEQ ID NO: 4 - protein, 495 aa, unidentified
  REGION 1..495: note = source = /note="Description of Unknown:LysK lysin sequence"
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKKTAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 5 - protein, 339 aa, unidentified
  REGION 1..339: note = source = /note="Description of Unknown:Cpl-1 lysin sequence"
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 6 - protein, 126 aa, synthetic construct
  REGION 1..126: note = source = /note="Description of Artificial Sequence: Syntheticpolypeptide"
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYR

SEQ ID NO: 7 - protein, 147 aa, synthetic construct
  REGION 1..147: note = source = /note="Description of Artificial Sequence: Syntheticpolypeptide"
MKISLEGLSLIKKFEGCKLEAYKCSAGVWTIGYGHTAGVKEGDVCTQEEAEKLLRGDIFKFEEYVQDSVKVDLDQSQFDALVAWTFNLGPGNLRSSTMLKKLNNGEYESVPFEMRRWNKAGGKTLDGLIRRRQAESLLFESKEWHQV

SEQ ID NO: 8 - protein, 144 aa, synthetic construct
  REGION 1..144: note = source = /note="Description of Artificial Sequence: Syntheticpolypeptide"
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 9 - protein, 181 aa, synthetic construct
  REGION 1..181: note = source = /note="Description of Artificial Sequence: Syntheticpolypeptide"
MNNELPWVAEARKYIGLREDTSKTSHNPKLLAMLDRMGEFSNESRAWWHDDETPWCGLFVGYCLGVAGRYVVREWYRARAWEAPQLTKLDRPAYGALVTFTRSGGGHVGFIVGKDARGNLMVLGGNQSNAVSIAPFAVSRVTGYFWPSFWRNKTAVKSVPFEERYSLPLLKSNGELSTNEA

SEQ ID NO: 10 - protein, 439 aa, synthetic construct
  REGION 1..439: note = source = /note="Description of Artificial Sequence: Syntheticpolypeptide"
MKRTTLNLELESNTDRLLQEKDDLLPQSVTNSSDEGTPFAQVEGASDDNTAEQDSDKPGASVADADTKPVDPEWKTITVASGDTLSTVFTKAGLSTSAMHDMLTSSKDAKRFTHLKVGQEVKLKLDPKGELQALRVKQSELETIGLDKTDKGYSFKREKAQIDLHTAYAHGRITSSLFVAGRNAGLPYNLVTSLSNIFGYDIDFALDLREGDEFDVIYEQHKVNGKQVATGNILAARFVNRGKTYTAVRYTNKQGNTSYYRADGSSMRKAFIRTPVDFARISSRFSLGRRHPILNKIRAHKGVDYAAPIGTPIKATGDGKILEAGRKGGYGNAVVIQHGQRYRTIYGHMSRFAKGIRAGTSVKQGQIIGYVGMTGLATGPHLHYEFQINGRHVDPLSAKLPMADPLGGADRKRFMAQTQPMIARMDQEKKTLLALNKQR

SEQ ID NO: 11 - protein, 39 aa, synthetic construct
  REGION 1..39: note = source = /note="Description of Artificial Sequence: Syntheticpolypeptide"
NKGDYQGAADQFPRWVNAGGKRLDGLVKRRASQSRESQC

SEQ ID NO: 12 - protein, 42 aa, synthetic construct
  REGION 1..42: note = source = /note="Description of Artificial Sequence: Syntheticpolypeptide"
NKGDYQGAADQFPRWVNAGGKRLDGLVKRRAAERALFLEPLS

SEQ ID NO: 13 - protein, 31 aa, synthetic construct
  REGION 1..31: note = source = /note="Description of Artificial Sequence: Syntheticpolypeptide"
NKGDYQGAADQFPRWVNAGGKRLDGLVKRRA

SEQ ID NO: 14 - protein, 31 aa, synthetic construct
  REGION 1..31: note = source = /note="Description of Artificial Sequence: Syntheticpolypeptide"
NKGDYQGAADQFPRWVNAGGKRLDGLVKRRK

SEQ ID NO: 15 - protein, 43 aa, synthetic construct
  REGION 1..43: note = source = /note="Description of Artificial Sequence: Syntheticpolypeptide"
NKGDYQGAADQFPRWVNAGGKRLDGLVKRRAAERALFLEPLSC

SEQ ID NO: 16 - protein, 495 aa, unidentified
  REGION 1..495: note = Description of Unknown:PAL lysin peptide
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

(19) United States
(12) Patent Application Publication
(10) Pub. No.: US 2025/0064902 A1
(43) Pub. Date: Feb. 27, 2025

(54) USE OF LYSIN TO RESTORE/AUGMENT ANTIBACTERIAL ACTIVITY IN THE PRESENCE OF PULMONARY SURFACTANT OF ANTIBIOTICS INHIBITED THEREBY

(71) Applicant: AUROBAC THERAPEUTICS SAS, Lyon (FR)

(72) Inventors: Michael WITTEKIND, Bainbridge Island, WA (US); Raymond SCHUCH, Mountain Lakes, NJ (US)

(73) Assignee: AUROBAC THERAPEUTICS SAS, Lyon (FR)

(21) Appl. No.: 18/914,723

(22) Filed: Oct. 14, 2024

Related U.S. Application Data
(63) Continuation of application No. 17/749,252, filed on May 20, 2022, now Pat. No. 12,144,845.
Application No. 17/749,252 is a division of application No. 15/760,416, filed on Mar. 15, 2018, now Pat. No. 11,357,833.
(60) Provisional application No. 62/220,212, filed on Sep. 17, 2015.
(60) Provisional application No. 62/247,619, filed on Oct. 28, 2015.

(51) Int. Cl. A61K 38/47; A61K 9/00; A61K 31/7036; A61K 38/12; A61K 38/16; A61K 38/46; A61K 45/06; A61P 31/04; C12N 9/36

(52) U.S. Cl. CPC A61K 38/47; A61K 9/0019; A61K 9/0073; A61K 31/7036; A61K 38/12; A61K 38/162; A61K 38/46; A61K 45/06; A61P 31/04; C12N 9/2462; C12Y 302/01017; A61K 2121/00; Y02A 50/30

(57) ABSTRACT

The present disclosure relates to methods for restoring or augmenting bactericidal activity of an antibiotic in an organ or tissue in which pulmonary surfactant is present. More specifically, the present disclosure describes that inhibition of antibiotics due to environmental factors, such as the presence of pulmonary surfactant in an organ or tissue such as the respiratory epithelium can be sidestepped or overcome and the effectiveness of the antibiotic in that milieu restored or augmented by co-administration of an antibiotic and a lysin.

### STATEMENT OF RELATED APPLICATIONS

This patent application is a continuation of U.S. application Ser. No. 17/749,252 filed May 20, 2022, which is a divisional application of U.S. patent application Ser. No. 15/760,416 (now issued U.S. Pat. No. 11,357,833), filed Mar. 15, 2018, which is a national phase application of International Patent Application No. PCT/US2016/052348, filed Sep. 16, 2016, and which claims the priority of U.S. Provisional Patent Application 62/220,212 filed Sep. 17, 2015, and U.S. Provisional Patent Application 62/247,619 filed Oct. 28, 2015; the contents of these provisional applications are hereby incorporated by reference in their entirety.

### SEQUENCE LISTING

This application contains a Sequence Listing which has been submitted electronically in XML format and is hereby incorporated by reference in its entirety. Said XML copy, created on 8 Oct. 2024, is named 0497.0012-02_SL.xml and is 25,336 bytes in size

### BACKGROUND OF THE DISCLOSURE

### Technical Field

The present disclosure relates to the field of combatting bacterial infection, specifically of the respiratory tract and more specifically of the lower respiratory tract, notably tissues and organs the epithelium of which is characterized by the presence of pulmonary surfactant. The disclosure more addresses a problem of reduced effectiveness of antibiotics in combatting infection due to factors in the environment of the infection, such as the pulmonary surfactant, rather than to antibiotic resistance developments.

### Description of the Related Art

Bacteriophage lysin polypeptide CF-301 is a first-in-class antimicrobial agent under development to treat Staphylococcus aureus bacteremia and endocarditis. Hallmark features of CF-301 include rapid pathogen-specific bacteriolysis, an absence of resistance, synergy with standard-of-care antibiotics and anti-biofilm activity (Schuch et al., J Infect Dis.;209(9):1469-78 (2014). doi: 10.1093/infdis/jit637. Epub 2013 Nov. 28.). CF-301 is the first lysin to enter FDA-regulated clinical trials. CF-301 (PlySs2) has the amino acid sequence depicted in SEQ ID NO: 1 (GenBank accession ZP 03625529) and has been described in U.S. Pat. No. 9,034,322.

Other lysins active against Staphylococci responsible for airway or respiratory tract infections include without limitation PlyC, PlyGBS, LysK, lysostaphin, chimeric lysin ClyH, (Cheng et al. Antimicrob Agents Chemother. 49(1):111-117 (2005); McGowan et al. Proc Natl Acad Sci U S A., 109(31): 12752-7 (2012), Becker et al. FEMS Microbiol Lett., 287(2):185-91(2008), Yang et al. Antimicrob Agents Chemother. 2014;58(1):536-42 (2014). Lysin polypeptides active against Streptococcus pneumoniae include PAL and Cpll lysins described respectively in WO 2008/00132 (including the sequence of the CHAP domain for chimerization) and CN 102021161 (Garcia et al. J Virol. 61(8):2573-80 (1987); Varca et al. J Biol Chem.,279(42):43697-707. (2004)). The disclosures of the foregoing patents and references are incorporated by reference in their entirety for all purposes. Several other lysins active against a variety of bacterial pathogens, including bacteria responsible for infections of the airways and more particularly the lower respiratory tract, have been identified.

The cyclic lipopeptide antibiotic daptomycin has been approved for skin and skin structure infections. Daptomycin is rapidly bactericidal against gram-positive (G+) bacteria and it exerts its activity by insertion into and disruption of the functional integrity of the G+ plasma membrane, a mechanism strongly dependent on the presence of physiologic levels of free calcium. However, daptomycin has failed to meet criteria in a clinical trial for severe community-acquired pneumonia. This deficiency has been shown to be due to an interaction between daptomycin and pulmonary surfactant, which inhibits the activity of this antibiotic specifically in the lung environment and more generally in the airway environment wherein pulmonary surfactant is present. Surfactant Inhibition of Daptomycin, Silverman, J. A. et al, JID, 191:2149-2152 (2005). Thus, daptomycin is not indicated for treatment of lung and more generally airway (especially lower respiratory tract) infections and those of skill in the art would not employ a treatment regimen including daptomycin to treat such infections. The inability of daptomycin to combat infection in the presence of pulmonary surfactants been shown dramatically in Koplowicz et al. Clin Infect Dis. 49(8):1286-7 (2009). Recent studies have focused on overcoming daptomycin inactivity in the presence of surfactant by testing and evaluating antibacterial activity of hybrid molecules of the structurally related lipopeptide A54145 (Nguyen et al. Antimicrob Agents Chemother. 2010 April; 54(4):1404-1413.)

Pulmonary surfactant, a primary component of epithelial lining fluid, is a complex lipid-and-protein mixture that coats the interior surface of the airway, reducing surface tension within the alveoli. Surfactant is composed primarily of dipalmitoylphosphatidylcholine (˜80% in all mammalian species), along with significant amounts of phosphatidylglycerol (PG) and smaller amounts of minor phospholipids, neutral lipids, and cholesterol. There are 4 protein components: hydrophilic proteins SP-A and SP-D and hydrophobic proteins SP-B and SP-C. Goerke J. Pulmonary surfactant: functions and molecular composition. Biochim Biophys Acta 1998; 1408:79-89. Daptomycin is inserted into artificial membrane vesicles composed of phosphatidylcholine (PC) and PC/PG. Lakey JH, et al: Fluorescence indicates a calcium-dependent interaction between the lipopeptide antibiotic LY146032 and phospholipid membranes. Biochemistry 1988; 27:4639-45; Jung D, et al. Structural transitions as determinants of the action of the calcium-dependent antibiotic daptomycin. Chem Biol 2004; 11:949-57.

A major problem in medicine has been the development of drug resistant bacteria as more antibiotics are used for a wide variety of illnesses and other conditions. Hospital infections are the 8^{th} leading cause of death in the United States, due in large part to drug-resistant and newly-emerging pathogens. For example, there are over 500,000 cases of Staphylococcus aureus annually in the U.S. and over 65% of strains are multidrug resistant (for example certain strains of methicillin-resistant S. aureus (MRSA) are also multidrug resistant. The use of more antibiotics and the number of bacteria showing resistance has prompted longer treatment times. Furthermore, broad, non-specific antibiotics, some of which have detrimental effects on the patient, are now being used more frequently. A related problem with this increased use is that many antibiotics do not penetrate mucus linings casily, or are inhibited by factors present in these linings as discussed above. Additionally, the number of people allergic to antibiotics appears to be increasing. Accordingly, there is a commercial need for new antibacterial approaches, especially those that operate via new modalities or provide new or improved means to kill pathogenic bacteria and thereby treat infection.

The discovery of lysin polypeptides, enzymes derived from bacteriophage that can penetrate the bacterial wall or outer membrane and directly lyse bacteria or expose them to bactericidal agents, such as the host's immune system and/or antibiotics, has been a breakthrough in the field of infectious disease. In particular, lysins administered in conjunction with antibiotics have been found to synergize with them, resulting in an increase in the effectiveness of antibiotics against even resistant pathogens. This synergy has opened the way for use of reduced doses of the antibiotic and/or the lysin, reducing the potential for side effects. See, e.g., U.S. Pat. No. 9,034,322.

However, where an antibiotic has been found ineffective in treating a particular infection caused by an otherwise susceptible pathogen because of environmental factors, such as surfactant inhibition, rather than resistance, the use of lysins has not been previously proposed. Indeed, there was no reason to expect that lysins would improve effectiveness of antibiotics in the face of inhibition by pulmonary surfactant. Accordingly, the effectiveness of the methods disclosed below was unexpected.

Gram-positive bacteria are surrounded by a cell wall containing polypeptides and polysaccharide. The gram-positive cell wall appears as a broad, dense wall that is 20-80 nm thick and consists of numerous interconnecting layers of peptidoglycan. Between 60% and 90% of the gram-positive cell wall is peptidoglycan, providing cell shape, a rigid structure, and resistance to osmotic shock. The cell wall does not exclude the Gram stain crystal violet, allowing cells to be stained purple, and therefore “Gram-positive.” Gram-positive bacteria include but are not limited to the genera Actinomyces, Bacillus, Listeria, Lactococcus, Staphylococcus, Streptococcus, Enterococcus, Mycobacterium, Corynebacterium, and Clostridium. Medically relevant species include Streptococcus pyogenes, Streptococcus pneumoniae, Staphylococcus aureus, and Enterococcus faecalis. Bacillus species, which are spore-forming, cause anthrax and gastroenteritis. Spore-forming Clostridium species are responsible for botulism, tetanus, gas gangrene and pseudomembranous colitis. Corynebacterium species cause diphtheria, and Listeria species cause meningitis. Staphylococcus aureus and Streptococcus pneumoniae are two major causative agents for pneumonia, whether community-acquired, nosocomial, secondary to aspiration or opportunistic.

Thus, to the extent that otherwise effective antibiotics are inhibited by factors present in the organ or tissue that is the site of the infection, such as pulmonary surfactant in the case of infections of the lungs or other airways and more generally of the respiratory tract, a treatment regimen that would restore and even augment activity of such antibiotics would be of great commercial and public health value.

In addition to daptomycin discussed above, other antibiotics that are known to be inhibited by pulmonary surfactant include without limitation: tobramycin, an aminoglycoside used to treat infections caused by the gram-negative bacterium Pseudomonas aeruginosa a common cause of pneumonia (van 't Veen A et al. Antimicrob. Agents Chemother. 39:329-333 (1995)), and colistin, a cyclic lipopeptide (polymixin) broadly active against gram-negative bacteria, including P. aeruginosa. Schwameis, R. et al, Effect of Pulmonary Surfactant on Antimicrobial Activity In Vitro, October 2013 Volume 57 Number 10 Antimicrobial Agents and Chemotherapy p. 5151-5154.

### SUMMARY OF THE DISCLOSURE

In one aspect, the present disclosure relates to a method for treating a subject afflicted with a bacterial infection of an organ or tissue in which pulmonary surfactant is present, the method comprising regardless of order the following steps:
a. administering to the subject a first amount of an antibiotic having antibacterial activity against the bacteria responsible for the infection which activity is inhibited by the pulmonary surfactant;
b. co-administering to the subject a second amount of a lysin polypeptide wherein said first and second amount in combination are effective to kill the bacteria responsible for the infection and thereby treat the infection.

In some embodiments, the lysin has antibacterial activity against the bacteria responsible for the infection.

In some embodiments, the first amount is such that it would be ineffective to treat the infection if the antibiotic were administered as monotherapy.

In some embodiments, the antibiotic is a cyclic lipopeptide or an aminoglycoside.

In a more particular embodiment the lysin polypeptide has the amino acid sequence of SEQ ID NO: 1 or variants thereof having antibacterial activity against Staphylococcus aureus and at least 80% sequence identity to SEQ ID NO: 1, and the bacterium responsible for the infection is Staphylococcus aureus.

In some embodiments the S. aureus is MRSA MSSA or VISA.

In some embodiments, the antibiotic is a cyclic lipopeptide, for example daptomycin.

In other embodiments the antibiotic is an aminoglucoside, for example tobramycin.

In some embodiments, the second amount or the first amount is a subthreshold amount (or both amounts are subthreshold).

In some embodiments, the lysin polypeptide is administered parenterally or by inhalation; in some embodiments, the antibiotic is administered orally or parenterally or by inhalation.

In some embodiments, the subject is a mammalian subject.

In some embodiments, the lysin polypeptide is PAL or Cpl-1 and the bacterium responsible for the infection is Streptococcus pneumoniae.

In some embodiments, the bacterium responsible for the infection is gram-negative, for example, P. aeruginosa.

In some embodiments, lysin is an artilysin described in one or more of the following patent applications: U.S. Pat. No. 20,140,120074, WO/2015/070912; WO/2015/071436; WO/2015/070911; WO/2015/071437; U.S. Pat. No. 20,150,118731 and WO/2012/085259 or is a GN lysin having a sequence selected from the group of Artilysins, described in one or more of the following patent applications: U.S. Pat. No. 20,140,120074, WO/2015/070912; WO/2015/071436; WO/2015/070911; WO/2015/071437; U.S. Pat. No. 20,150,118731 and WO/2012/085259 and the following gram-negative lysins disclosed in U.S. Provisional Patent Application 62/247,619 filed Oct. 28, 2015, copy of which is attached to this patent application as Appendix A, and which is incorporated by reference in its entirety: GN37 (SEQ ID NO: 6); GN2 (SEQ ID NO: 7); GN4 (SEQ ID NO: 8); GN14 (SEQ ID NO: 9); GN43 (SEQ ID NO: 10); PGN4 (SEQ ID NO: 11); FGN4-1 (SEQ ID NO: 12); FGN4-2 (SEQ ID NO: 13); FGN4-3 (SEQ ID NO: 14); and FGN4-4 (SEQ ID NO: 15).

In various more specific embodiments, the antibiotic is a cyclic lipopeptide such as colistin or an aminoglycoside such as tobramycin.

In another aspect, the disclosure relates to a method for treating a subject afflicted with a streptococcus or staphylococcus bacterial infection of the lower respiratory tract in which pulmonary surfactant is present, the method comprising regardless of order the following steps:
a. administering to the subject a first amount of an antibiotic having antibacterial activity against the bacteria responsible for the infection which activity is inhibited by the pulmonary surfactant;
   i. co-administering to the subject a second amount of at least one lysin polypeptide, selected from the group consisting of: CF-301, ClyS, lysostaphin, LysK, Sal-200, LysGH15, PlyV12, ClyH, MV-L, Ply, PlyPly, PlyGBS, LambdaSal, LambdaSa2, Cpl1, Pal, active fragments thereof, and chimeric combinations thereof wherein the binding domain of one of the foregoing lysins or fragments has been fused to the catalytic domain of another
wherein said first and second amount in combination are effective to kill the bacteria responsible for the infection and thereby treat the infection.

In some embodiments, the antibiotic is daptomycin.

A method for restoring or augmenting bactericidal activity of an antibiotic in an organ or tissue in which pulmonary surfactant is present in an amount that is or would be inhibitory of the activity of the antibiotic against a bacterial infection in said organ or tissue, the method comprising: administering to a subject afflicted with an infection of said organ or tissue a first amount of said antibiotic and co-administering to the subject a second amount of a lysin polypeptide having antibacterial activity against the bacterium responsible for the infection, wherein administration of the lysin overcomes or sidesteps the inhibition, the amounts in combination being effective to kill said bacterium and thereby treat the infection.

### BRIEF DESCRIPTION OF THE DRAWINGS

FIGS. 1A-1C demonstrate that CF-301 is active in bovine-derived surfactant while DAP is not active. FIG. 1A shows MIC values for CF-301 and DAP against MRSA strain MW2, FIG. 1B shows MIC values for CF-301 and DAP against MSSA strain ATCC 29213, and FIG. 1C shows MIC values for CF-301 and DAP against VISA strain ATCC 700699.

FIG. 2 includes images demonstrating that CF-301 promotes BODIPY-DAP (DAPBD) binding to MRSA in 7.5% surfactant. Mag: 1000×

FIG. 3 includes images demonstrating that CF-301 promotes DAP^{BD} binding to VISA in 7.5% surfactant. Mag=2000×.

FIGS. 4A-4B include TEM (FIG. 4A) and SEM (FIG. 4B) analysis images demonstrating that CF-301 and DAP act together to kill S. aureus and reduce biofilm-like structures in 7.5% surfactant. In FIG. 4A, scale bars are 0.5 μm. In FIG. 4B, scale bars are 2 μm (5,000× images) and 1 μm (20,000× images).

FIG. 5A is a survival curve of mice infected intranasally with 5×10⁸ CFUs of S. aureus (MRSA strain ATCC BAA-42) and treated with saline, CF-301 (i.v.), DAP (s.c.), or the CF-301/DAP. (n=10 mice/group; p<0.05 vs. DAP). FIG. 5B is a plot of Log of CFU/lungs 1 and 3 days post infection.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### Definitions

The following terms and phrases include the meanings provided below unless the context clearly indicates otherwise.

The term “treatment” refers to any process, action, application, therapy, or the like, wherein a subject, including a human being, is subjected to medical aid with the object of providing a treatment for or curing a disorder, or killing or cradicating a pathogen, or improving the subject's condition, directly or indirectly. Treatment also refers to reducing incidence, or alleviating symptoms, eliminating recurrence, preventing recurrence, preventing incidence, improving symptoms, improving prognosis or combinations thereof. “Treatment” further encompasses reducing the population, growth rate or virulence of the bacteria in the subject and thereby controlling or reducing a bacterial infection in a subject or bacterial contamination of an organ or tissue or environment. Thus “treatment” that reduces incidence is effective to inhibit growth of at least one Gram-positive or of at least one Gram-negative bacterium in a particular milieu, whether it be a subject or an environment. On the other hand “treatment” of an already established infection or contamination refers to reducing the population or killing, including even eradicating Gram-positive or Gram-negative bacteria responsible for an infection or contamination.

“Preventing” includes the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease is reduced, and such constitute examples of prevention. Contracted diseases in the context of the present disclosure encompass both those manifesting with clinical or subclinical symptoms, such as the detection of as well as the detection of growth of a bacterial pathogen when symptoms associated with such pathologyare not yet manifest.

The term “effective amount” refers to an amount which, when applied or administered in an appropriate frequency or dosing regimen, is sufficient to prevent or inhibit bacterial growth or prevent, reduce or ameliorate the onset, severity, duration or progression of the disorder being treated (here bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” is intended to embrace separate administration of a lysin polypeptide and an antibiotic or any other antibacterial agent in a sequential manner as well as administration of these agents in a substantially simultaneous manner, such as in a single mixture/composition or in doses given separately, but nonetheless administered substantially simultaneously to the subject, for example at different times in the same day or 24-hour period (or in a shorter or longer interval as long as the administration of the antibiotic benefits from the conjoint administration of the lysin). Such co-administration of lysin polypeptides with one or more additional antibacterial agents such as antibiotics can be provided as a continuous treatment lasting up to days, weeks or months. Additionally, the co-administration need not be continuous or co-extensive as long as the inhibition of the administered antibiotic by pulmonary surfactant is abated and effectiveness of the antibiotic in treating infections of an organ or tissue wherein pulmonary surfactant is present is restored or augmented.

“Subject” refers to a subject to be treated and includes inter alia a mammal, including without limitation a human, a plant, a lower animal, a single cell organism or a cell culture. For example, the term “subject” is intended to include organisms, e.g., prokaryotes and eukaryotes, which are susceptibe to or afflicted with Gram-negative or Gram-positive bacterial infections. Examples of subjects include mammals, c.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to a bacterial infection, whether such infection be systemic or confined to a particular organ or tissue.

“Polypeptide” is used interchangeably with the term “protein” and “peptide” and refers to a polymer made from amino acid residues and having at least about 30 amino acid residues. The term includes not only polypeptides in isolated form, but also active fragments and derivatives thereof (defined below). The term “polypeptide” also encompasses fusion proteins or fusion polypeptides comprising a lysin polypeptide as described below and maintaining the lysin function. A polypeptide can be a naturally occurring polypeptide or an engineered or synthetically produced polypeptide. A particular lysin polypeptide can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, as illustrated for example herein with a fragment of GN4 comprising the amphipathic domain of GN4 and further truncated versions thereof maintaining lysin activity against the same or at least one common target bacterium (see Appendix A). Variants of native lysin polypeptides are also encompassed having at least 80% or at least 85% or at least 90% or at least 95% or at least 98% sequence identity with the native lysin polypeptide (which, as stated above includes active fragments of a native lysin protein).

“Bactericidal” in the context of an agent or a compound conventionally means having the property of causing the death of bacteria or capable of killing bacteria to an extent of at least a 3-log (99.9%) or better reduction among an initial population of bacteria.

“Augmenting” within the context of the present disclosure means that a degree of antimicrobial activity of an antibiotic is higher than it would be in the presence of pulmonary surfactant. For example, antibiotic activity in the context of the present disclosure can be restored or augmented by at least 5 fold, at least 10 fold, at least 16 fold, at least 20 fold, at least 24 fold, at least 30 fold, at least 40 fold, at least 50 fold, at least 70 fold, at least 80 fold at least 100 fold, more than 10 fold, more than 20 fold, more than 50 fold, more than 100 fold. Additionally, in the context of the present disclosure, the activity of lysin can be augmented by at least 2 fold, at least 4 fold, at least 8 fold, at least 10 fold, up to 10 fold, up to 16 fold, up to 20 fold, more than 2 fold, more than 4 fold, more than 8 fold, more than 10 fold, more than 20 fold.

“Inhalable” refers to a method of direct delivery of a composition to the respiratory tract during or in conjunction with routine or assisted respiration (e.g., by intratracheobronchial, pulmonary, and/or nasal administration). Inhalable formulations include, but are not limited to atomized, nebulized, dry powder and/or aerosolized formulations.

“Biofilm” refers to an aggregate of bacteria that are embedded within a self-produced matrix of polysaccharides, glycoproteins or nucleic acids. In this state, bacteria are highly resistant to antibiotics.

### Embodiments

In some embodiments, the present disclosure describes combining CF-301 with the antibiotic daptomycin (DAP) to expand the indications for both drugs to infections of an organ or tissue, such as infections of the airways, wherein pulmonary surfactant is present. In some embodiments, the pulmonary surfactant is expressed in organs or tissues other than respiratory system (Madsen et al. Am J Respir Cell Mol Biol., 29(5):591-7 (2000)). While DAP is a potent therapeutic option for bacteremia and endocarditis, it cannot be used for pulmonary infections because of selective inhibition by pulmonary surfactant (Silverman et al., J Infect Dis., 191(12):2149-52. (2005)). In light of clinical limitations associated with surfactant-mediated inhibition of DAP, the present disclosure describes that CF-301 restores or augments DAP activity in the lung (and other portions of the respiratory tract wherein pulmonary surfactant is present including for example the bronchial passages but also the trachea and pharynx), wherein DAP activity is normally inhibited by pulmonary surfactant and as such offers a new option for treating airway and notably lower respiratory tract infections, such as staph pneumonia, bronchial pneumonia, pneumococcal pneumonia and atypical pneumonia.

More broadly, the present disclosure describes that inhibition of antibiotics due to environmental factors, such as the presence of pulmonary surfactant in an organ or tissue such as the respiratory epithelium can be sidestepped or overcome and the effectiveness of the antibiotic in that milieu restored or augmented by co-administration of an antibiotic and a lysin.

The antibiotic may be one to which the causative agent of the infection to be treated is normally susceptible; the lysin may be one which is active against the same organism. Typically, the antibiotic will be administered in a first amount, such as one which would be an effective amount when used as monotherapy in the absence of the surfactant or a smaller amount including in certain embodiments a subthreshold amount, since the antibiotic will be substantially freed from interference by the surfactant and available to synergize with the lysin. Thus the antibiotic amount to be employed will be subject to fine-tuning which is well within the skill of the art. The lysin will typically be administered in a second amount, such as one that would be employed if the lysin were used as monotherapy, or a smaller amount, including in certain embodiments a subthreshold amount since the lysin and the antibiotic synergize. Again, the amount of the lysin will be subject to optimization which is well within the skill of the art. The first and second amounts will be such that at least in combination (if not also individually) will be effective to kill bacteria responsible for the infection and thereby treat the infection, thereby eradicating it or contributing to its partial or complete eradication.

In one embodiment, the lysin is administered in a first amount, and the antibiotic is administered in a second amount.

The antibiotic may be administered by any appropriate route, such as parenteral, oral or in certain cases by inhalation. The lysin may be administered by any appropriate route, by injection (parenterally) or by inhalation. The duration of therapy will be determined by assessment of the effectiveness of the treatment, such as by the attenuation and/or disappearance of symptoms, the reduction or elimination of pathogen titers, the improvement in the physical condition of the treated subject, etc., as well as by the rate of improvement in one or more of such assessment parameters. There may well be variation from subject to subject depending on such factors as age, type of infection, attending complications and general physical condition of the patient. The normal duration of antibiotic monotherapy will be a bench mark for determining the duration of the conjoint therapy according to the present disclosure.

Due to the presence of pulmonary surfactant, the interior of the airway has a unique environment within the body. Studies have shown that in certain instances, organ-specific inhibition of an antibiotic can occur, resulting in inefficacy of a particular antibiotic in that specific organ. Such organ-specific inhibition has been observed in the case of daptomycin (DAP), wherein small amounts of pulmonary surfactant were capable of inhibiting DAP activity against Staphylococcus aureus, rendering DAP not suitable for the treatment of pulmonary infections caused by this pathogen (Silverman et al., J Infect Dis., 191(12):2149-52.(2005)). Studies by Silverman et al. were further corroborated in a patient treated with DAP for bronchoalveolar pneumonia due to S. aureus (Koplowicz et al. Clin Infect Dis. 49(8):1286-7 (2009)). Both studies (Silverman et al., and Koplowicz et al.) established that the presence of pulmonary surfactants hampers the antimicrobial action of DAP. Based on this, it is anticipated that DAP will be active and available to treat infections that are due to other respiratory pathogens provided that DAP is active against such pathogens in the absence of pulmonary surfactant (e.g., in vitro or when the infection is established in an organ or tissue devoid or substantially devoid of pulmonary surfactant). Nonlimiting examples of such pathogens are coagulase negative staphylococci, Streptococcus pneumoniae and Streptococcus pyogenes.

In addition to DAP, which belongs to a class of cyclic lipopeptide antibiotics, pulmonary surfactant-induced inhibition of antibiotic activity has been observed for additional antibiotics, such as colistin, a lipopetide, and tobramycin, an aminoglycoside. Thus, the methods of the present disclosure can be used to restore or augment activity of these antibiotics against susceptible bacterial pathogens, wherein such pathogens infect an organ or tissue where pulmonary surfactant is present.

Currently, DAP is indicated for the treatment of complicated skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcus faecalis (vancomycin-susceptible isolates only). DAP is also used in the treatment of Staphylococcus aureus bloodstream infections, including those with right-sided infective endocarditis, caused by methicillin-susceptible and methicillin-resistant isolates. Furthermore, in vitro studies have shown that penicillin resistant Streptococcus pneumoniae is inhibited by DAP (Piper et al. J Infect Chemother (2005) 11:207-209).

In one embodiment, the present disclosure provides methods for restoring or augmenting surfactant-inhibited antibiotic activity comprising administering a combination of a lysin and one or more antibiotic to an organ or a tissue wherein pulmonary surfactant is present. Since pulmonary surfactants are present in lung tissue, and the present disclosure provides in vitro and in vivo evidence of lysin's ability to restore the antimicrobial activity of surfactant-inhibited antibiotic, it is anticipated that other bacteria that cause infections of the lower respiratory tract will be killed by combinations of DAP and lysins active against these bacteria.

It shall be understood that the lysins exemplified herein including in Tables 1 through 3 can be replaced by active fragments thereof and chimeric combinations of the binding domain of one lysin with the catalytic domain of another. See, e.g., Cheng et al. Appl Microbiol Biotechnol. 74(6):1284-91 (2007). Indeed some of the examples are already fragments or chimeric lysin polypeptides.

TABLE 1
Examples of: bacteria susceptible to DAP treatment (in the absence of pulmonary surfactant),
types of infection that occur in the presence of pulmonary surfactant, and lysin compound(s)
capable of killing or inhibiting the growth of each bacteria listed.

| | | Lysin capable of inhibiting |
|---|---|---|
| Bacteria | Type of Infection | the growth of said bacteria |
| Staphylococcus | respiratory tract infections, | CF-301 (SEQ ID NO: 1), |
| aureus | pneumonia | ClyS (SEQ ID NO: 2), |
| | | lysostaphin, LysK (SEQ ID |
| | | NO: 4) [GenBank: |
| | | AFN38929.1], Sal-200 and |
| | | LysGH15 (which are |
| | | derivatives of LysK), PlyV12 |
| | | (SEQ ID NO: 3), ClyH, and |
| | | MV-L[GenBank: |
| | | AB254389.1]. |
| Streptococcus | infections of the upper respiratory | CF-301 (Gilmer et al. |
| pyogenes | tract, pneumonia (Steer et al. | Antimicrob. Agents |
| | Drugs. 2012 Jun. 18; | Chemother. June 2013 vol. |
| | 72(9): 1213-27). | 57 no. 6 2743-2750) |
| | | PlyC (Nelson et al. Proc Natl |
| | | Acad Sci USA. 2006 Jul. 11; |
| | | 103(28): 10765-10770.) |
| | | PlyGBS (Cheng et al. |
| | | Antimicrob Agents |
| | | Chemother. 2005 January; 49(1): |
| | | 111-117.), PlyGBS mutants |
| | | (Cheng et al. Appl Microbiol |
| | | Biotechnol. 74(6): |
| | | 1284-91 (2007) |
| | | PlyPly (Lood et al. |
| | | Antimicrob Agents |
| | | Chemother. 2014 June; 58(6): |
| | | 3073-3084.) |
| Streptococcus | infections of the upper respiratory | CF-301 (Schuch et al. J |
| agalactiae | tract, pneumonia | Infect Dis. 2014 May 1; |
| | | 209(9): 1469-1478.) |
| | | LambdaSa1, LambdaSa2 |
| | | (Pritchard et al. Appl Environ |
| | | Microbiol. 2007 November; 73(22): |
| | | 7150-7154. |
| Streptococcus | Infections of the upper respiratory | PlySK1249 (Oechslin et al. |
| dysgalactiae subsp. | tract, pneumonia (Preziuso et al. J | Antimicrob Agents |
| equisimilis | Vet Sci. 2010 March; 11(1): 67-72.) | Chemother. 2013 December; |
| | | 57(12): 6276-6283) |
| vancomycin- | respiratory infections, pneumonia | PlyV12 (SEQ ID NO: 3) |
| resistant | | |
| Enterococcus | | |
| faecalis | | |
| Streptococcus | respiratory infections, pneumonia | Cpl-1 (SEQ ID NO : 5) |
| pneumoniae | | [NC_001825.1], dimerized |
| | | forms of Cpl-1, Pal (Fenton |
| | | et al. Bioeng Bugs. 2010 January- |
| | | February; 1(1): 9-16.) |

The entire disclosure of all documents cited in the above table are incorporated by reference in their entirety for all purposes.

The aminoglycoside class of antibiotics comprises many different agents. Gentamicin, tobramycin, amikacin, streptomycin, neomycin, and paromomycin are approved by the US Food and Drug Administration (FDA). Tobramycin is active against various Gram-negative bacteria, including, but not limited to P. aeruginosa, E. coli, Acinetobacter spp., Citrobacter spp., Enterobacter spp. and other. In particular, tobramycin displays high activity against P. aeruginosa, a common causative agent of pneumonia, both community acquired and nosocomial.

In terms of Gram-positive bacteria, tobramycin exhibits a narrower spectrum of activity, wherein with the exception of S. aureus and S. epidermidis, most Gram-positive bacteria are resistant to tobramycin. However, similar to DAP, tobramycin activity against Klebsiella pneumoniae, Pseudomonas aeruginosa, S. aureus, and S. pneumoniae is reduced in the presence of surfactant (van 't Veen A et al. Antimicrob. Agents Chemother. 39:329-333(1995)). Infections associated with Klebsiella pneumoniae, Pseudomonas aeruginosa, S. aureus, and S. pneumoniae and lysins active against these bacteria are listed in Table 2.

Thus, the methods of the present disclosure can be used for restoring or augmenting surfactant-inhibited antibiotic activity in order to treat infections caused by Gram positive bacteria, or Gram negative bacteria, or both. Commonly, infections are polymicrobial, with mixed Gram-positive and Gram-negative species (Citron et al. J Clin Microbiol. 45(9):2819-2828 (2007)). In some embodiments, the methods of the present disclosure can be used for restoring or augmenting surfactant-inhibited antibiotic activity in order to treat a polymicrobial infection.

TABLE 2
Examples of: bacteria susceptible to tobramycin treatment (in the
absence of surfactant), types of infection that occur in the presence
of pulmonary surfactant, and lysin compound(s) capable of killing
or inhibiting the growth of each bacterium listed.

| | | Lysin capable of |
|---|---|---|
| | | inhibiting the growth of |
| Bacteria | Infection | said bacteria |
| Klebsiella | pneumonia; lower | Artilysins, described in one |
| pneumoniae | respiratory tract | or more of the following |
| | infections | patent applications: |
| | | US 20140120074, |
| | | WO/2015/070912; |
| | | WO/2015/071436; |
| | | WO/2015/070911; |
| | | WO/2015/071437; |
| | | US 20150118731 and |
| | | WO/2012/085259 |
| | | GN37 (SEQ ID NO: 6) |
| | | GN2 (SEQ ID NO: 7) |
| | | GN4 (SEQ ID NO: 8) |
| | | GN14 (SEQ ID NO: 9) |
| | | GN43 (SEQ ID NO: 10) |
| | | PGN4 (SEQ ID NO: 11) |
| | | FGN4-1 (SEQ ID NO: 12) |
| | | FGN4-2 (SEQ ID NO: 13) |
| | | FGN4-3 (SEQ ID NO: 14) |
| | | FGN4-4 (SEQ ID NO: 15) |
| Pseudomonas | respiratory system | Artilysins, described in one |
| aeruginosa, | infections, pneumonia | or more of the following |
| | | patent applications: |
| | | US 20140120074, |
| | | WO/2015/070912; |
| | | WO/2015/071436; |
| | | WO/2015/070911; |
| | | WO/2015/071437; |
| | | US 20150118731 and |
| | | WO/2012/085259 |
| | | Also, the following Gram |
| | | negative lysins identified by |
| | | the present inventors: |
| | | GN37 (SEQ ID NO: 6) |
| | | GN2 (SEQ ID NO: 7) |
| | | GN4 (SEQ ID NO: 8) |
| | | GN14 (SEQ ID NO: 9) |
| | | GN43 (SEQ ID NO: 10) |
| | | PGN4 (SEQ ID NO: 11) |
| | | FGN4-1 (SEQ ID NO: 12) |
| | | FGN4-2 (SEQ ID NO: 13) |
| | | FGN4-3 (SEQ ID NO: 14) |
| | | FGN4-4 (SEQ ID NO: 15) |
| S. aureus | respiratory system | CF-301, ClyS (SEQ ID |
| | infections, pneumonia | NO: 2), lysostaphin, LysK |
| | | (SEQ ID NO: 4), Sal-200 |
| | | and LysGH15 (which are |
| | | derivatives of LysK), |
| | | PlyV12 (SEQ ID NO: 3), |
| | | ClyH (Yang et al. |
| | | Antimicrob Agents |
| | | Chemother. 2014 January; |
| | | 58(1): 536-542) |
| S. pneumoniae | respiratory infections, | Cpl-1 (SEQ ID NO: 5) |
| | pneumonia | (including dimerized form |
| | | of Cpl-1), Pal (SEQ |
| | | ID NO: 16) |

The entire disclosure of all documents cited in the above table are incorporated by reference in their entirety for all purposes.

Colistin (also known as polymyxin E) belongs to the polymyxin group of antibiotics. Colistin has a narrow antibacterial spectrum and is primarily used for infections with P. aeruginosa and A. baumannii. Infections associated with P. aeruginosa and A. baumanni and lysins active against these bacteria are listed in Table 3.

Table 3. examples of bacteria susceptible to tobramycin treatment (in the absence of surfactant), type of infection that occurs in the presence of pulmonary surfactant, and lysin compound(s) capable of killing or inhibiting the growth of each bacteria listed.

| | | Lysin capable of inhibiting |
|---|---|---|
| Bacteria | Infection | the growth of said bacteria |
| P. aeruginosa | respiratory system | Artilysins, described in one |
| | infections, pneumonia | or more of the following |
| | | patent applications: |
| | | US 20140120074, |
| | | WO/2015/070912; |
| | | WO/2015/071436; |
| | | WO/2015/070911; |
| | | WO/2015/071437; |
| | | US 20150118731 and |
| | | WO/2012/085259 |
| | | In addition the following |
| | | lysins identified by the |
| | | present inventors can be |
| | | used. |
| | | GN37 (SEQ ID NO: 6) |
| | | GN2 (SEQ ID NO: 7) |
| | | GN4 (SEQ ID NO: 8) |
| | | GN14 (SEQ ID NO: 9) |
| | | GN43 (SEQ ID NO: 10) |
| | | PGN4 (SEQ ID NO: 11) |
| | | FGN4-1 (SEQ ID NO: 12) |
| | | FGN4-2 (SEQ ID NO: 13) |
| | | FGN4-3 (SEQ ID NO: 14) |
| | | FGN4-4 (SEQ ID NO: 15) |
| A. baumannii | respiratory infection, | PlyF307 [ [GenBank: |
| | pneumonia | KJ740396.1] |

The entire disclosure of all documents cited in the above table are incorporated by reference in their entirety for all purposes.

Pulmonary infection due to S. aureus can occur among individuals either in the community or in a hospital setting. Furthermore, pulmonary infection due to S. aureus can develop among individuals with S. aureus colonization of the skin or nares. Often, the infection due to S. aureus occurs in the context of intubation or other respiratory tract instrumentation. S. aureus pneumonia can also occur following viral pneumonia or in the setting of right-sided endocarditis with pulmonary emboli.

The most common causes of bacterial lung infections in normal hosts include Streptococcus pneumoniae, Haemophilus species, Staphylococcus aureus, and Mycobacterium tuberculosis.

The primary cause of morbidity and mortality in patients with cystic fibrosis (CF) is bronchiectasis and obstructive lung disease. Pulmonary disease is present in 98% of patients with CF by the time they reach adulthood. Despite the great advances in the management of this disorder, the majority of the patients succumb to respiratory complications. S aureus is one of the pathogens most commonly found in the airways of patients with CF. Thus, in one embodiment, the present disclosure is directed to treatment of S. aureus pulmonary infection in subjects with CF by administering daptomycin and a lysin active against the pathogen, such as CF-301.

As stated above, the present disclosure provides methods for restoring or augmenting surfactant-inhibited antibiotic activity comprising administering a combination of a lysin, and one or more antibiotic to an organ or a tissue wherein pulmonary surfactant is present.

In one embodiment, the present disclosure provides a method of treatment of a subject afflicted with a bacterial infection of an organ or tissue in which pulmonary surfactant is present, such as the lung or more generally the respiratory tract, comprising administering to the subject a first amount of an antibiotic that is normally inhibited by pulmonary surfactant and co-administering to the subject a second amount of a lysin polypeptide wherein the first and second amounts are together effective to treat the infection (this statement does not preclude the individual components of a combination having an effect of their own). The lysin preferably targets, i.e., it is active against, the bacteria responsible for the infection. The pathogens responsible for the infection may be resistant to at least one standard of care antibiotic but must be susceptible to the antibiotic used in the combination with lysin.

In another embodiment, the present disclosure provides a method of treatment of a subject afflicted with a bacterial infection of an organ or tissue in which pulmonary surfactant is present, such as the lung or more generally the respiratory tract, comprising administering to the subject a first amount of a lysin polypeptide and co-administering to the subject a second amount of an antibiotic that is normally inhibited by pulmonary surfactant wherein the first and second amounts are together effective to treat the infection (this statement does not preclude the individual components of a combination having an effect of their own).

In another embodiment, the infection of the airway is a staphylococcal related disease or condition (e.g., a disease or condition associated with presence of Staphylococcus bacteria including those diseases resulting from staphylococcus infection or staphylococcus infection is sequela to another disease or condition, such as a transplant or cancer or cancer therapy such as chemotherapy).

In some embodiments, the present disclosure provides a method for restoring or augmenting bactericidal activity of an antibiotic in a subject afflicted with a bacterial infection of an organ or tissue in which pulmonary surfactant is present in an amount that is or would be inhibitory of the activity of the antibiotic against a bacterial infection in said subject, the method comprising: administering to a subject afflicted with an infection of said organ or tissue a first amount of said antibiotic and co-administering to the subject a second amount of a lysin polypeptide having antibacterial activity against the bacterium responsible for the infection, the amounts in combination being effective to kill said bacterium and thereby treat the infection.

The present disclosure further provides methods for restoring or augmenting lysin activity, such as CF-301, comprising administering a combination of antibiotic and lysin (e.g., DAP and CF-301 lysin). In an aspect thereof, the activity of lysin CF-301 lysin is enhanced at least 2 fold, at least 4 fold, at least 8 fold, at least 10 fold, up to 10 fold, up to 16 fold, up to 20 fold, or more.

### EXAMPLES

### Example 1

### CF-301, but not DAP, is Active in Pulmonary Surfactant

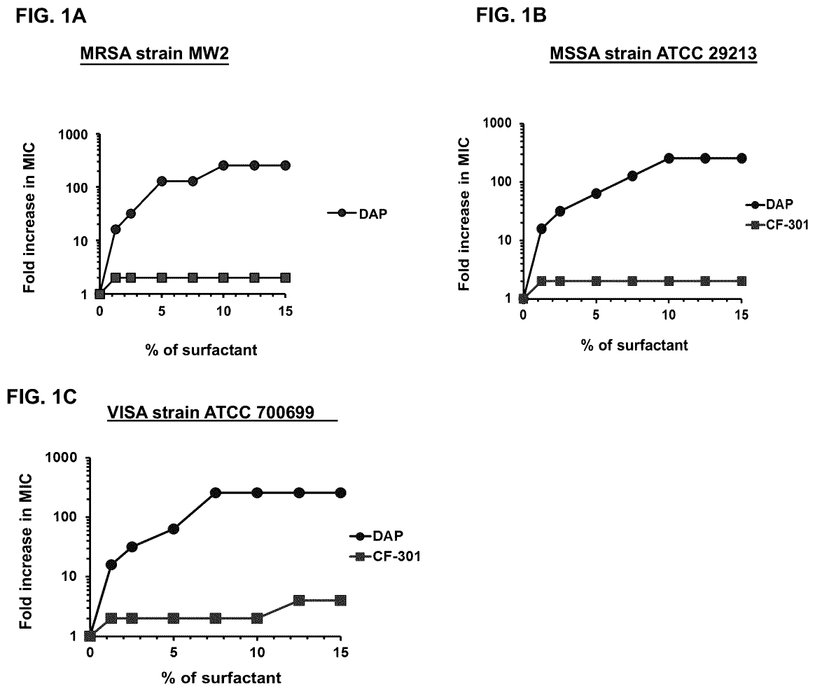

In order to determine individual activity of CF-301 and DAP against different strains of Staphylococcus aureus in the presence of surfactant, the inventors tested 3 different S. aureus strains and used bovine-derived surfactant (Survanta, AbbVie Inc), which is a functional equivalent of human surfactant. Minimum inhibitory concentration (MIC) determination was preformed using methicillin resistant strain (MRSA) MW2 (FIG. 1A), methicillin-susceptible (MSSA) strain ATCC 29213 (FIG. 1B), and vancomycin-intermediate staphylococcus aureus (VISA) strain ATCC 700699 (FIG. 1C), in the presence of increasing concentrations of surfactant (FIGS. 1A-1B). MIC values were determined by broth microdilution according to Clinical and Laboratory Standards Institute. M07-A9. Methods for dilution antimicrobial susceptibility tests for bacteria that grow aerobically; approved standard. 8th ed. Wayne, PA: CLSI, 2012. Briefly, each strain of bacteria was suspended in growth media using calcium-adjusted Mueller-Hinton broth at the concentration of 5×10⁵ colony-forming units [CFU] /mL and exposed to CF-301 or DAP in a series of 2-fold serial dilutions in 96-well polypropylene microtiter plates (Becton, Dickinson, and Company). Following 24 hours of incubation at 35° C. in ambient air, MIC values were recorded as the most dilute concentration of each compound (CF-301 or DAP) that inhibited bacterial growth of each strain (Schuch et al, J Infect Dis.; 209 (9): 1469-78 (2014)). Starting MIC values (i.e., without surfactant) for CF-301 and DAP (respectively) were 32 and 1 μg/ml for MW2 (FIG. 1A), 16 and 1 μg/ml for ATCC 29213 (FIG. 1B) and 64 and 2 μg/ml for ATCC 700699 (FIG. 1C).

As shown in FIG. 1A-C, CF-301, but not DAP, showed antimicrobial activity in the presence of pulmonary surfactant in each strain tested. CF-301 MIC increased up to 2-fold (for a MRSA and MSSA strain) and 4-fold (for VISA) over a range of surfactant concentrations from 1.25-15% (FIGS. 1A-C). DAP MIC however, increased 256-fold over the same range of surfactant as the range used for CF-301 study. Collectively, these results show that CF-301 active in the presence of surfactant against MRSA, MSSA, and VISA strains of S. aureus, while DAP is not active.

### Example 2

DAP Activity in Pulmonary Surfactant is Permitted when used in Combination with CF-

Following the findings that DAP is not active in the presence of surfactant, the inventors sought to evaluate the possibility that CF-301 promotes DAP activity in the presence of surfactant and allows DAP to excert its antimicrobial function even in the presence of a pulmonary surfactant. Antimicrobial activity of CF-301 and DAP together in the presence of surfactant was assessed using 2 different methods: combination MIC assay and the checkerboard assay. The checkerboard dilution test is widely used method for testing of in vitro synergy between multiple compounds (White et al. Antimicrob Agents Chemother. 40(8):1914-8 (1996)). Checkerboards were generated using combinations of sub-MIC CF-301 with sub-MIC daptomycin against a panel of 20 MRSA and 20 MSSA strains in 7.5% surfactant. Combination MIC assay is a variation of the microdilution method, whereby two compounds in combination (rather than a single compound) are diluted two-fold across the x-axis of a 96 well plate (Schuch et al, J Infect Dis.; 209 (9): 1469-78 (2014)) and the lowest concentration of the compound combination (in this instance CF-301 and DAP) required to inhibit growth of bacteria is determined. For purpose of experimental design, synergy was defined as inhibitory activity greater than what would be predicted by adding the 2 compounds together (ie, minimum fractional inhibitory concentration [FICmin]≤0.5) (Moody J. 2007. Synergism testing: broth microdilution checkerboard and broth macrodilution methods, p 1-23 In Garcia L S, Isenberg HD, editors. (ed), Clinical microbiology procedures handbook, 2nd ed. ASM Press, Washington, DC).

As shown in Table 4, combining CF-301 and DAP in the presence of 7.5% surfactant resulted in growth inhibitory concentrations 16-32-fold and 512-1024-fold lower, respectively, than when each compound was used as single agent. Importantly, combining CF-301 and DAP restored the activity of DAP despite the presence of a surfactant, indicating that the addition of lysin to otherwise surfactant-inhibited antibiotic overcomes the inhibition of such antibiotics. The results were consistent among various strains, including 5 strains of MRSA (MW2, BAA-1720, NRS-192, NRS-265, NRS-255) and 5 strains of MSSA (ATCC-29213, NRS-131, ATCC 25923, ATCC 49521, and Newman) (Table 4, data are MIC values for each drug alone and in combination.)).

Furthermore, as evident by the checkerboard assay using MHB supplemented with 7.5% surfactant, sub-MIC concentrations of CF-301 and DAP exhibited potent synergy (FIC≤0.5) against a panel of 20 MRSA and MSSA strains (Tables 5 and 6). For values listed in Tables 2 and 3, individual MICs and combination fractional inhibitory concentrations (FICs) are shown, wherein FIC values ≤0.5 indicate strong synergy.

Taken together, these results demonstrate that CF-301 restores and promotes DAP activity in the presence of pulmonary surfactant.

TABLE 4
Combining CF-301 and DAP restores DAP activity on the
presence of surfactant and CF-301 and DAP are highly active
together against S. aureus in 7.5% surfactant.

| | | CF-301 | | | Daptomycin | | |
|---|---|---|---|---|---|---|---|
| | | MIC | MIC | Fold | MIC | MIC | Fold |
| | Strain | alone | combo | reduction | alone | combo | reduction |
| MRSA | MW2 | 64 | 2 | 32 | 256 | 0.25 | 1024 |
| | BAA-1720 | 64 | 2 | 32 | 256 | 0.25 | 1024 |
| | NRS-192 | 64 | 4 | 16 | 256 | 0.5 | 512 |
| | NRS-265 | 64 | 4 | 16 | 256 | 0.25 | 1024 |
| | NRS-255 | 64 | 2 | 16 | 512 | 0.5 | 1024 |
| MSSA | ATCC 29213 | 128 | 4 | 32 | 256 | 0.5 | 512 |
| | NRS-131 | 64 | 4 | 16 | 512 | 0.5 | 1024 |
| | ATCC 25923 | 128 | 4 | 32 | 256 | 0.5 | 512 |
| | ATCC 49521 | 64 | 2 | 32 | 256 | 0.5 | 512 |
| | Newman | 128 | 4 | 32 | 256 | 0.5 | 512 |

TABLE 5
CF-301 synergizes with DAP against
MRSA isolates in 7.5% surfactant.

| CFS# (strain name) | CF-301 MIC | DAP MIC | FIC |
|---|---|---|---|
| 269 (MW2) | 64 | 256 | 0.375 |
| 223 (BAA-1720) | 64 | 256 | 0.375 |
| 738 (NRS-192) | 64 | 256 | 0.312 |
| 735 (NRS-265) | 64 | 256 | 0.500 |
| 743 (NRS-255) | 64 | 256 | 0.375 |
| 218 (BAA-42) | 64 | 256 | 0.375 |
| 836 (BAA-1688) | 128 | 256 | 0.312 |
| 958 (JMI-227) | 128 | 256 | 0.375 |
| 962 (JMI-1004)* | 64 | 256 | 0.500 |
| 981 (JMI-3346)* | 64 | 256 | 0.312 |

*Respiratory isolate

TABLE 6
CF-301 synergizes with DAP against
MSSA isolates in 7.5% surfactant.

| CFS# (strain name) | CF-301 MIC | DAP MIC | FIC |
|---|---|---|---|
| 554 (ATCC 25923) | 128 | 256 | 0.5 |
| 581 (ATCC 29213) | 128 | 256 | 0.5 |
| 919 (ATCC 49521) | 64 | 256 | 0.5 |
| 28 (Newman) | 128 | 256 | 0.5 |
| 258 (NRS-153) | 128 | 256 | 0.5 |
| 766 (NRS-106) | 64 | 256 | 0.375 |
| 757 (NRS-131) | 64 | 256 | 0.375 |
| 960 (JMI-316)* | 128 | 256 | 0.312 |
| 964 (JMI-1040)* | 128 | 256 | 0.5 |
| 966 (JMI-1173)* | 128 | 256 | 0.375 |

*Respiratory isolate

### Example 3

CF-301 Promotes (or Allows) DAP Binding to Staphylococcus aureus

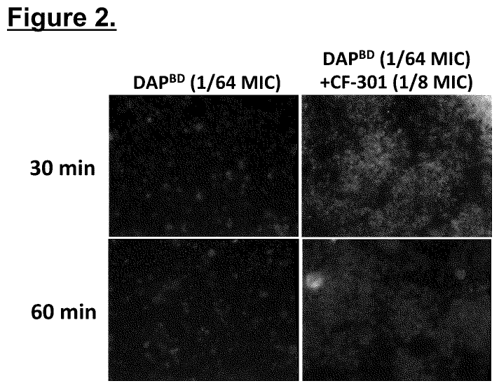

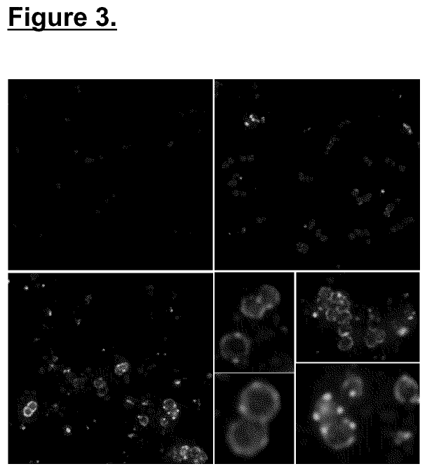

Given that CF-301 restores the activity of DAP in the presence of surfactant (Example 2), the inventors postulated that CF-301 promotes DAP binding to Staphylococcus aureus. BODIPY-labeled daptomycin (BDP-DAP) assay was used to assess the interaction of DAP with the bacterial cell membrane (CM), as described before (Tran et al. MBio., 23;4 (4) (2013)). Briefly, mid-log phase MRSA MW2 (FIG. 2) and VISA ATCC 700699 (FIG. 3) strain cells were stained with DAPI, washed, and resuspended in 25 mM Tris pH 7.2 with 50μg/ml CaCl₂ and 7.5% surfactant. The BODIPY-DAP was then added (to 4 μg/ml), followed by CF-301 (to 4 or 8 μg/ml). A control contained no CF-301. After incubation for either 30 or 60 minutes at room temperature, cells were diluted, washed, fixed and plated on 0.01% lysine coated slides before visualization by fluorescence microscopy (FIG. 2, 1000x; FIG. 3, Mag=2000x).

As shown in FIG. 2, CF-301 (8 μg/ml) promoted BODIPY-DAP (DAP^{BD}) binding to MRSA in the presence of 7.5% surfactant. Similarly, CF-301 (4 μg/m) promoted DAP^{BD} binding to VISA in 7.5% surfactant (FIG. 3, VISA strain ATCC 700699 labeled with DAPI and treated 30 min with buffer (FIG. 3, panel A), DAP^{BD} (4 μg/ml; 1/64 MIC) (FIG. 3, panel B), or DAP^{BD} and CF-301 (4 μg/ml; 1/128 MIC) (FIG. 3, panels C-G).). Collectively, these findings indicate that CF-301 promotes DAP binding to bacterial CM.

### Example 4

CF-301 and DAP act together to kill S. aureus and reduce/disrupt biofilm-like structures in 7.5% surfactant.

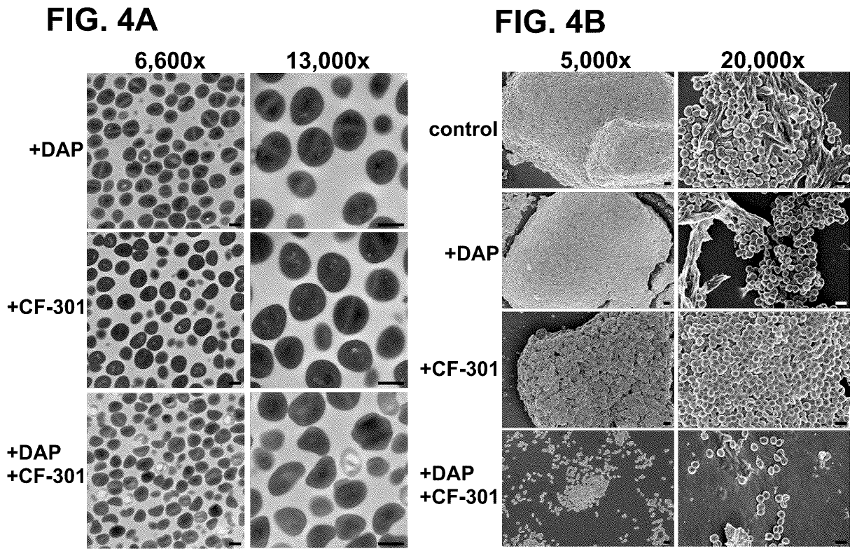

Next, the inventors investigated the ability of CF-301 and DAP together to kill S. aureus and reduce and disrupt the biofilm-like structures in the presence of surfactant in 25 mM Tris pH7.2 (with 50 μg/ml CaCl2 and 7.5% surfactant). VISA strain ATCC 700699 was treated for 20 min alone (control) or with DAP (4 μg/ml; 1/64 MIC), CF-301 (4 μg/ml; 1/128 MIC), or the combination of DAP and CF-301 Transmission electron microscopy (TEM) (FIG. 4A) and scanning electron microscopy (SEM) (FIG. 4B) analysis indicate the efficient killing of S. aureus (FIG. 4A), as well as the reduction in biofilm formation (FIG. 4B) when CF-301 and DAP were combined. Thus, similarly to what was observed in prior examples, these observations indicate that CF-301 allows DAP to overcome inhibitory effects of surfactant.

### Example 5

Combination Therapy with CF-301 and DAP is Superior to Monotherapy in a Murine Model of S. aureus Pneumonia

Considering the advantages observed in vitro when CF-301 and DAP were combined, the effects of using CF-301 and DAP together in vivo were evaluated. In order to address this question, mice were infected intranasally with 5×10⁸ CFUs of S. aureus (MRSA strain ATCC BAA-42) and treated with saline, CF-301 (i.v.), DAP (s.c.), or the CF-301/DAP combination once daily beginning four hours after the start of infection (n=10 mice/group; p<0.05 vs. DAP). The experiment was carried out for 14 days post infection. At 14 days, treatment with the CF-301 and DAP combination resulted in 70% survival, demonstrating that combination therapy was superior to either drug alone (P<0.05 vs. DAP).

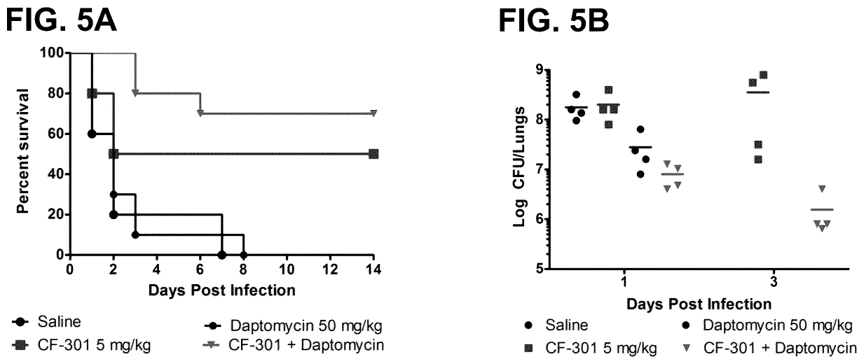

As shown in FIG. 5A, combination therapy with CF-301 and DAP was superior to monotherapy in a murine model of S. aureus pneumonia. Similar to what was observed in vitro, use of CF-301 in addition to DAP results in the restoration of DAP antimicrobial activity. In vivo data obtained here further supports those findings. For example, animals treated with DAP alone exhibit same survival pattern as those treated with saline (control). However, addition of CF-301 to DAP treatment restores the antimicrobial activity of CF-301.

Furthermore, the total number of bacterial CFUs in the lungs of each of 4 infected animals groups (measured 1 and 3 days post infection) was significantly reduced after the treatment with CF-301 and DAP combined (FIG. 5B).

As shown by the Examples described herein, CF-301 promotes DAP activity and permits its antimicrobial effects to be carried out in the presence of surfactant. These findings were corroborated both in vitro and in vivo.

In summary, the inventors have used minimum (and in some experiments sub-minimum) inhibitory concentration (MIC) and checkerboard assays with and without bovine pulmonary surfactant (functional equivalent of human surfactant), to show a potent synergistic interaction between CF-301 and DAP against MRSA, MSSA, and VISA Staphylococcus aureus isolates. MIC reductions of up to 1024-fold were observed for DAP in the presence of CF-301 in surfactant. Furthermore, efficacy of CF-301 and/or DAP was demonstrated in a BALB/c mouse lung infection model following survival and CFU levels. The in vitro and in vivo results shown in Examples 1-5 suggest that CF-301 combination with DAP could be an effective therapy targeting S. aureus lung infections.

CF-301 synergizes with DAP—at sub-MIC levels—to kill a range of MSSA and MRSA isolates in the presence of pulmonary surfactant (a potent inhibitor of DAP). The results show a more rapid accumulation of DAP within bacterial cells in the presence of CF-301. Significantly, the combination therapy is highly efficacious in the lung environment of infected mice, suggesting that CF-301 and DAP is effective at treating staphylococcal pneumonia, a new indication for both drugs. The complementary and synergistic activities of these agents are reinforced by the novel features of CF-301, which includes rapid bacteriolysis, specificity for S. aureus, the absence of resistance, and potent anti-biofilm activity.

All references cited herein are incorporated by reference in their entirety for all purposes. The foregoing examples are illustrative and nonlimiting. While specific embodiments are described above, those of skill in the art will readily be able to envision additional embodiments, modifications and variations all within the scope of the claims set forth below including equivalents.

```
Sequence ID NO: 1 (CF-301, GenBank Accession Number: ZP_03625529)
ATGACAACAG TAAATGAAGC ATTAAATAAT GTAAGAGCTC AGGTTGGGTC
CGGTGTGTCT GTTGGCAACG GCGAATGCTA CGCTTTGGCT AGTTGGTACG
AGCGCATGAT TAGTCCGGAT GCAACTGTCG GACTTGGGGC TGGTGTGGGC
TGGGTCAGCG GTGCAATCGG CGATACAATC TCTGCCAAAA ACATCGGCTC
ATCATACAAC TGGCAAGCTA ACGGCTGGAC AGTTTCCACA TCTGGTCCAT
TTAAAGCAGG TCAGATTGTG ACGCTTGGGG CAACACCAGG AAACCCTTAC
GGACATGlGG TAATCGTCGA AGCAGTGGAC GGCGATAGAT TGACTATTTT
GGAGCAAAAC TACGGGGGGA AACGTTATCC CGTCCGTAAT TATTACAGCG
CTGCAAGCTA TCGTCAACAG GTCGTGCATT ACATCACACC GCCTGGCACG
GTCGCACAGT CAGCACCCAA CCTTGCAGGC TCTCGTTCCT ATCGCGAGAC
GGGCACTATG ACTGTCACGG TCGATGCTCT CAATGTTCGC AGGGCGCCAA
ATACTTCAGG CGAGATTGTA GCAGTATACA AGCGTGGlGA ATCATTTGAC
TATGATACTG TCATCATCGA TGTCAATGGC TATGTCTGGG TGTCTTACAT
AGGCGGCAGC GGCAAACGTA ACTACGTTGC GACGGGCGCT ACCAAAGACG
GTAAGCGTTT CGGCAATGCT TGGGGTACAT TTAAATAA
Sequence ID NO: 2 (ClyS)
Met Glu Thr Leu Lys Gln Ala Glu Ser Tyr Ile Lys Ser Lys Val Asn 1 5 10
            15
Thr Gly Thr Asp Phe Asp Gly Leu Tyr Gly Tyr Gln Cys Met Asp Leu 20 25 30
Ala Val Asp Tyr Ile Tyr His Val Thr Asp Gly Lys Ile Arg Met Trp 35 40 45
Gly Asn Ala Lys Asp Ala Ile Asn Asn Ser Phe Gly Gly Thr Ala Thr 50 55 60
Val Tyr Lys Asn Tyr Pro Ala Phe Arg Pro Lys Tyr Gly Asp Val Val 65 70 75
              80
Val Trp Thr Thr Gly Asn Phe Ala Thr Tyr Gly His Ile Ala Ile Val 85 90 95
Thr Asn Pro Asp Pro Tyr Gly Asp Leu Gln Tyr Val Thr Val Leu Glu 100 105 110
Gln Asn Trp Asn Gly Asn Gly Ile Tyr Lys Thr Glu Leu Ala Thr Ile 115 120 125
Arg Thr His Asp Tyr Thr Gly Ile Thr His Phe Ile Arg Pro Asn Phe 130 135 140
Ala Thr Glu Ser Ser Val Lys Lys Lys Asp Thr Lys Lys Lys Pro Lys 145 150 155
              160
Pro Ser Asn Arg Asp Gly Ile Asn Lys Asp Lys Ile Val Tyr Asp Arg 165 170 175
Thr Asn Ile Asn Tyr Asn Met Val Leu Gln Gly Lys Ser Ala Ser Lys 180 185 190
Ile Thr Val Gly Ser Lys Ala Pro Tyr Asn Leu Lys Trp Ser Lys Gly 195 200 205
Ala Tyr Phe Asn Ala Lys Ile Asp Gly Leu Gly Ala Thr Ser Ala Thr 210 215 220
Arg Tyr Gly Asp Asn Arg Thr Asn Tyr Arg Phe Asp Val Gly Gln Ala 225 230 235
              240
Val Tyr Ala Pro Gly Thr Leu Ile Tyr Val Phe Glu Ile Ile Asp Gly 245 250 255
Trp Cys Arg Ile Tyr Trp Asn Asn His Asn Glu Trp Ile Trp His Glu 260 265 270
Arg Leu Ile Val Lys Glu Val Phe 275
SEQ ID NO: 3 (PlyV12)
MTRRYTKMNVPQSLVNWFVNHRNLLTYSMYGSRNGSDGTADCSGSMSQAL
KEAGlPIQGLPSTVTLGQQLAKNGFYRISRNEDWNAETGDIVLMSWGADMAS
SGGAGGHVGVMMDSVNFISCDYSTQGAAGQAINTYPWNDYYEANKPAYIEV
WRYSESAPQTKNQANTAVTPQQKAYYEANEVKYVNGlWQIKCDYLSPIGFD
YLENGlPVTMVNWVDKDGNDLPDGADQDLKAGMYFSFSSDETNIVDTGNGG
YYGGYYWRLFEFGQFGPVWLSCWNKDDLVNYFQ
SEQ ID NO: 4 (LysK)
MAKTQAEINKRLDAYAKGTV DSPYRVKKAT SYDPSFGVME AGAIDADGYY
HAQCQDLITD YVLWLTDNKV RTWGNAKDQI KQSYGTGFKI HENKPSTVPK
KGWIAVFTSG SYEQWGHIGlVYDGGNTSTF TILEQNWNGY ANKKPTKRVD
NYYGLTHFIE IPVKAGTTVK KKTAKKSASK TPAPKKKATL KVSKNHINYT
MDKRGKKPEGMVIHNDAGRSSGQQYENSLA NAGYARYANG IAHYYGSEGY
VWEAIDAKNQ IAWHTGDGTG ANSGNFRFAG IEVCQSMSAS DAQFLKNEQA
VFQFTAEKFK EWGLTPNRKT VRLHMEFVPT ACPHRSMVLH TGFNPVTQGR
PSQAIMNKLK DYFIKQIKNY MDKGTSSSTV VKDGKTSSAS TPATRPVTGS
WKKNQYGTWY KPENATFVNG NQPIVTRIGS PFLNAPVGGN LPAGATIVYD
EVCIQAGHIW IGYNAYNGNR VYCPVRTCQG VPPNQIPGVA WGVFK
SEQ ID NO: 5 (Cpl-1)
MVKKNDLFVDVSSHNGYDITGlLEQMGTTNTIIKISESTTYLNPCLSAQVEQSN
PIGFYHFARFGGDVAEAEREAQFFLDNVPMQVKYLVLDYEDDPSGDAQANT
NACLRFMQMIADAGYKPIYYSYKPFTHDNVDYQQILAQFPNSLWIAGYGLND
GTANFEYFPSMDGlRWWQYSSNPFDKNIVLLDDEEDDKPKTAGTWKQDSKG
WWFRRNNGSFPYNKWEKIGGVWYYFDSKGYCLTSEWLKDNEKWYYLKDN
GAMATGWVLVGSEWYYMDDSGAMVTGWVKYKNNWYYMTNERGNMVSN
EFIKSG KGWYFMNTNG ELADNPSFTKEPDGLITVA
SEQ ID NO: 6
GN37
Polypeptide sequence
MTYTLSKRSLDNLKGVHPDLVAVVHRAIQLTPVDFAVIEGLRSVSRQKEL
VAAGASKTMNSRHLTGHAVDLAAYVNGlRWDWPLYDAIAVAVKAAAKELG
VAIVWGGDWTTFKDGPHFELDRSKYR
SEQ ID NO: 7
GN2
Polypeptide sequence
MKISLEGLSLIKKFEGCKLEAYKCSAGVWTIGYGHTAGVKEGDVCTQEEAEK
LLRGDIFKFEEYVQDSVKVDLDQSQFDALVAWTFNLGPGNLRSSTMLKKLNN
GEYESVPFEMRRWNKAGGKTLDGLIRRRQAESLLFESKEWHQV
SEQ ID NO: 8
GN4
Polypeptide sequence
MRTSQRGlDLIKSFEGLRLSAYQDSVGVWTIGYGTTRGVTRYMTITVEQAER
MLSNDIQRFEPELDRLAKVPLNQNQWDALMSFVYNLGAANLASSTLLKLLN
KGDYQGAADQFPRWVNAGGKRLDGLVKRRAAERALFLEPLS
SEQ ID NO: 9
GN14
Polypeptide sequence
MNNELPWVAEARKYIGLREDTSKTSHNPKLLAMLDRMGEFSNESRAWWHD
DETPWCGLFVGYCLGVAGRYVVREWYRARAWEAPQLTKLDRPAYGALVTF
TRSGGGHVGFIVGKDARGNLMVLGGNQSNAVSIAPFAVSRVTGYFWPSFWR
NKTAVKSVPFEERYSLPLLKSNGELSTNEA
SEQ ID NO : 10
GN43
Polypeptide sequence
MKRTTLNLELESNTDRLLQEKDDLLPQSVTNSSDEGTPFAQVEGASDDNTAE
QDSDKPGASVADADTKPVDPEWKTITVASGDTLSTVFTKAGLSTSAMHDML
TSSKDAKRFTHLKVGQEVKLKLDPKGELQALRVKQSELETIGLDKTDKGYSF
KREKAQIDLHTAYAHGRITSSLFVAGRNAGLPYNLVTSLSNIFGYDIDFALDL
REGDEFDVIYEQHKVNGKQVATGNILAARFVNRGKTYTAVRYTNKQGNTSY
YRADGSSMRKAFIRTPVDFARISSRFSLGRRHPILNKIRAHKGVDYAAPIGTPI
KATGDGKILEAGRKGGYGNAVVIQHGQRYRTIYGHMSRFAKGlRAGTSVKQ
GQIIGYVGMTGLATGPHLHYEFQINGRHVDPLSAKLPMADPLGGADRKRFM
AQTQPMIARMDQEKKTLLALNKQR
SEQ ID NO: 11
PGN4
Polypeptide sequence
NKGDYQGAADQFPRWVNAGGKRLDGLVKRRASQSRESQC
SEQ ID NO: 12
FGN4-1
Polypeptide Sequence
NKGDYQGAADQFPRWVNAGGKRLDGLVKRRAAERALFLEPLS
SEQ ID NO: 13
FGN4-2
Polypeptide Sequence
NKGDYQGAADQFPRWVNAGGKRLDGLVKRRA
SEQ ID NO: 14
FGN4-3
Polypeptide sequence
NKGDYQGAADQFPRWVNAGGKRLDGLVKRRK
SEQ ID NO: 15
FGN4-4
Polypeptide sequence
NKGDYQGAADQFPRWVNAGGKRLDGLVKRRAAERALFLEPLSC
Sequence ID 16: PAL Sequence
Met Ala Lys Thr Gln Ala Glu Ile Asn Lys Arg Leu Asp Ala Tyr Ala 1 5
                                    10 15
Lys Gly Thr Val Asp Ser Pro Tyr Arg Val Lys Lys Ala Thr Ser Tyr 20 25
                                        30
Asp Pro Ser Phe Gly Val Met Glu Ala Gly Ala Ile Asp Ala Asp Gly 35
                                                                    40 45
Tyr Tyr His Ala Gln cys Gln Asp Leu Ile Thr Asp Tyr Val Leu Trp 50
                                                        55 60
Leu Thr Asp Asn Lys Val Arg Thr Trp Gly Asn Ala Lys Asp Gln Ile 65
                                                70 75
                                                80
Lys Gln Ser Tyr Gly Thr Gly Phe Lys Ile His Glu Asn Lys Pro Ser 85
                                                                                      90 95
Thr Val Pro Lys Lys Gly Trp Ile Ala val Phe Thr Ser Gly Ser Tyr 100
                                                                                105 110
Glu Gln Trp Gly His Ile Gly Ile val Tyr Asp Gly Gly Asn Thr Ser
115 120 125
Thr Phe Thr Ile Leu Glu Gln Asn Trp Asn Gly Tyr Ala Asn Lys Lys 130
                                                        135 140
Pro Thr Lys Arg Val Asp Asn Tyr Tyr Gly Leu Thr His Phe Ile Glu 145
                                                150
                                                160
Ile Pro Val Lys Ala Gly Thr Thr val Lys Lys Glu Thr Ala Lys Lys 165
                                                                                        170 175
Ser Ala Ser Lys Thr Pro Ala Pro Lys Lys Lys Ala Thr Leu Lys Val 180
                                                                            185 190
Ser Lys Asn His Ile Asn Tyr Thr Met Asp Lys Arg Gly Lys Lys Pro 195
                                                                    200 205
Glu Gly Met Val Ile His Asn Asp Ala Gly Arg Ser Ser Gly Gln Gln 210
                                                            215 220
Tyr Glu Asn Ser Leu Ala Asn Ala Gly Tyr Ala Arg Tyr Ala Asn Gly 225
                                              230 235
                                              240
Ile Ala His Tyr Tyr Gly Ser Glu Gly Tyr Val Trp Glu Ala Ile Asp 245 250
                                                                                          255
Ala Lys Asn Gln Ile Ala Trp His Thr Gly Asp Gly Thr Gly Ala Asn 260
                                                                        265 270
Ser Gly Asn Phe Arg Phe Ala Gly Ile Glu Val Cys Gln Ser Met Ser 275
                                                                      280 285
Ala ser Asp Ala Gln Phe Leu Lys Asn Glu Gln Ala Val Phe Gln Phe 290
                                                          295 300
Thr Ala Glu Lys Phe Lys Glu Trp Gly Leu Thr Pro Asn Arg Lys Thr 305
                                                310 315
                                                320
Val Arg Leu His Met Glu Phe Val Pro Thr Ala Cys Pro His Arg Ser 325
                                                                                      330 335
Met Val Leu His Thr Gly Phe Asn Pro Val Thr Gln Gly Arg Pro
ser 340 345 350
Gln Ala Ile Met Asn Lys Leu Lys Asp Tyr Phe Ile Lys Gln Ile Lys
355 360 365
Asn Tyr, Met Asp Lys Gly Thr Ser Ser Ser Thr Val Val Lys Asp Gly
  370 3758 380
Lys Thr Ser Ser Ala Ser Thr Pro Ala Thr Arg Pro Val Thr Gly
Ser 385 390 395 400
Trp Lys Lys Asn Gln Tyr Gly Thr Trp Tyr Lys Pro Glu Asn Ala
  Thr 405 410 415
Phe val Asn Gly Asn Gln Pro Ile Val Thr Arg Ile Gly Ser Pro
Phe 420 425 430
Leu Asn Ala Pro Val Gly Gly Asn Leu Pro Ala Gly Ala Thr Ile
  Val 435 440 445
Tyr Asp Glu Val cys Ile Gln Ala Gly His Ile Trp Ile Gly Tyr
Asn 450 455 460
Ala Tyr Asn Gly Asn Arg Val Tyr cys Pro Val Arg Thr cys Gln
Gly 465 470 475 480
Val Pro Pro Asn Gln Ile Pro Gly Val Ala Trp Gly Val Phe
Lys 485 490 495
```

What is claimed is:

1. A method for treating a subject afflicted with a Gram-negative bacterial infection of an organ or tissue in which pulmonary surfactant is present, the method comprising regardless of order the following steps:
a. administering to the subject a first amount of an antibiotic having antibacterial activity against a Gram-negative bacteria responsible for the infection which activity is inhibited by the pulmonary surfactant;
b. co-administering to the subject a second amount of a lysin polypeptide,
wherein said first and second amount in combination are effective to kill the Gram-negative bacteria responsible for the infection and thereby treat the infection; and
wherein the lysin polypeptide comprises an amino acid sequence at least 80% identical to SEQ ID NO: 8 (GN4).

2. The method of claim 1, wherein the first amount is such that it would be ineffective to treat the infection if the antibiotic were administered as monotherapy.

3. The method of claim 1, wherein the antibiotic is a cyclic lipopeptide or an aminoglycoside.

4. The method of claim 3, wherein the antibiotic is a cyclic lipopeptide.

5. The method of claim 4 wherein the antibiotic is tobramycin.

6. The method of claim 1, wherein the second amount is a subthreshold amount.

7. The method of claim 1, wherein the first amount is a subthreshold amount.

8. The method of claim 1, wherein said lysin polypeptide is administered parenterally or by inhalation.

9. The method of claim 1, wherein said antibiotic is administered orally or parenterally of by inhalation.

10. The method of claim 1, wherein said subject is a mammalian subject.

11. The method of claim 1, wherein the Gram-negative bacteria is Pseudomonas aeruginosa.

12. The method of claim 1, wherein the antibiotic is colistin.

13. The method of claim 1, wherein the antibiotic is tobramycin.

14. A method for treating a subject afflicted with a Gram-negative bacterial infection of the lower respiratory tract in which pulmonary surfactant is present, which subject has already been administered an antibiotic suitable for treating the infection, the method comprising continuing administration of the antibiotic to the subject and commencing co-administration to the subject of a bactericidal activity-restoring amount of a lysin polypeptide having activity against the Gram-negative bacteria responsible for the infection in the subject and thereby restoring bactericidal activity of the antibiotic against the Gram-negative bacteria responsible for the infection of the lower respiratory tract of the subject,
wherein the lysin polypeptide comprises an amino acid sequence at least 80% identical to SEQ ID NO: 8 (GN4).

15. The method of of claim 14, wherein the lysin polypeptide does not require an exogenously-derived cationic peptide to have activity against the Gram-negative bacterial responsible for the infection.

16. The method of claim 15, wherein the lysin polypeptide further comprises at least one heterologous peptide segment covalently linked to the lysin polypeptide.

17. The method of claim 1, wherein the lysin polypeptide comprises an amino acid sequence at least 90% identical to SEQ ID NO: 8 (GN4).

18. The method of claim 17, wherein the lysin polypeptide comprises an amino acid sequence at least 95% identical to SEQ ID NO: 8 (GN4).

19. The method of claim 14, wherein the lysin polypeptide comprises an amino acid sequence at least 90% identical to SEQ ID NO: 8 (GN4).

20. The method of claim 19, wherein the lysin polypeptide comprises an amino acid sequence at least 95% identical to SEQ ID NO: 8 (GN4).
